(19) United States
(12) Patent Application Publication
(10) Pub. No.: US 2005/0245854 A1
(43) Pub. Date: Nov. 3, 2005

(54) Cervical brace

(75) Inventors: Eric C. Leuthardt, St. Louis, MO (US); Dennis J. Rivet, St. Louis, MO (US); Guy M. Genin, St. Louis, MO (US); Stuart P. Rosenberg, Wheeling, IL (US); Laura M. Seger, Leawood, KS (US); Elizabeth L. Tran, Opelousas, LA (US)

Correspondence Address: SONNENSCHEIN NATH & ROSENTHAL LLP, P.O. BOX 061080, WACKER DRIVE STATION, SEARS TOWER, CHICAGO, IL, 60606-1080, US

(73) Assignee: Washington University

(21) Appl. No.: 11/110,121

(22) Filed: Apr. 20, 2005

Related U.S. Application Data
(60) Provisional application No. 60/564,303, filed on Apr. 21, 2004.

(51) Int. Cl. A61F 5/00

(52) U.S. Cl. 602/18

(57) ABSTRACT

A cervical brace for restraining movement of a head and neck vertebra of a patient relative to at least one of a chest and an abdomen of the patient. The brace includes a mount sized and shaped for attachment to at least one of the chest and the abdomen of the patient and a support extending upward from the mount to a position in use adjacent a rearward surface of the head of the patient. The brace includes a loop sized and shaped for receiving the head of the patient cantilevered from the support. The support includes at least one joint for adjusting at least one of a pitch, a yaw and a roll of the loop relative to the mount.

This application claims priority from U.S. Provisional Patent Application No. 60/564,303 filed Apr. 21, 2004, entitled, “Low Profile, Cantilevered Halo Device”, which is hereby incorporated by reference.

### BACKGROUND OF THE INVENTION

The present invention relates generally to a cervical brace, and more particularly to a cervical brace having a cantilevered loop.

Cervical braces immobilize a patient's head to facilitate healing of a patient's upper spinal column after a fracture, a dislocation, or a surgery involving the upper spinal column. Generally, cervical braces restrain the head from rolling (such as occurs when a patient tilts her head from side to side), yawing (such as occurs when the patient shakes her head “no”), pitching (such as occurs when the patient nods her head “yes”). Conventional cervical braces immobilize the head by fixing the head to a stiff cast or vest positioned over a patient's torso. Usually, patients wear braces for three to six consecutive months. Afterwards, the patient may wear a soft collar (e.g., a Philadelphia collar) around the neck until the neck muscles regain strength.

Conventional cervical braces consist of a plaster body cast and a steel frame rigidly mounted on and extending upward from the cast. Typically, the frame includes four generally vertical rods positioned at 1:30, 4:30, 7:30 and 10:30 clock positions about the patient's head (measured from a 12:00 clock position at the front of the head) and a horizontal circular ring rigidly mounted on the rods at about forehead level. Pins extend inward from the ring to pierce skin and penetrate an outer layer of the skull to rigidly fix the ring to the patient's head. Thus, the head is rigidly held in a position relative to the patient's torso. Such conventional cervical braces allow portable stability and free jaw movement.

Conventional cervical braces such as described above have evolved little since their initial use. Many braces still include vests. The interiors of these vests are frequently lined with lambskin. Although the lambskin is compressed when the patient puts on the vest, the lambskin permits the vest to move relative to the torso and spine. Thus, even though these conventional devices constrain head movement relative to the vest, they less effectively constrain head movement relative to the torso and spine. Moreover, the frame, and the vertical rods in particular, obstruct the patient's view and protrude in such a way as to inhibit an ordinary lifestyle. For example, patients cannot wear most off-the-shelf shirts when wearing a conventional cervical brace because the rods interfere with their use. Although attempts have been made to reduce the motion of vests, the resulting cervical braces obstruct the patient's vision and are not low-profile so they interfere with an ordinary lifestyle. Moreover, prior cervical braces are time consuming and cumbersome to install due to the large number of screws needed at each adjustment point. Because cervical braces need frequent adjustment, the large number of screws further increases adjustment time. Even though conventional braces have a large number of screws, these braces are often particularly difficult to fit to patients with uneven shoulders. Still further, prior cervical braces have a ring or loop that circumscribes the head or a loop having an open portion in the back of the head. In either case, the loop extends in front of the patient's forehead, thereby further obstructing vision and interfering with an ordinary lifestyle.

### SUMMARY OF THE INVENTION

Briefly, the present invention includes a cervical brace for restraining movement of a head and neck vertebra of a patient relative to at least one of a chest and an abdomen of the patient. The brace comprises a mount sized and shaped for attachment to at least one of the chest and the abdomen of the patient and a support extending upward from the mount to a position in use adjacent a rearward surface of the head of the patient. In addition, the brace includes a loop sized and shaped for receiving the head of the patient cantilevered from the support. The support includes at least one joint for adjusting at least one of a pitch, a yaw and a roll of the loop relative to the mount.

In another aspect, the invention includes a cervical brace for restraining movement of a head and neck vertebra of a patient relative to at least one of a chest and an abdomen of the patient. The brace comprises a mount sized and shaped for attachment to at least one of the chest and the abdomen of the patient and a strap extending from the mount for attaching the mount to at least one of the chest and the abdomen of the patient. Further, the brace includes a support extending from the mount to a position in use adjacent a rearward surface of the head of the patient and a loop sized and shaped for receiving the head of the patient cantilevered from the support.

In yet another aspect, the invention includes a cervical brace for restraining movement of a head and neck vertebra of a patient relative to at least one of a chest and an abdomen of the patient. The brace comprises a mount sized and shaped for attachment to at least one of the chest and the abdomen of the patient and a support extending from the mount to a position in use adjacent a rearward surface of the head of the patient. The brace also includes a U-shaped loop sized and shaped for receiving the head of the patient cantilevered from the support. The loop is open at the front of the head of the patient in use.

Other features of the present invention will be in part apparent and in part pointed out hereinafter.

### BRIEF DESCRIPTION OF THE DRAWINGS

FIG. 1 is a front perspective of a cervical brace of one embodiment of the present invention;

FIG. 2 is a rear perspective of the cervical brace;

FIG. 3 is a rear perspective of the cervical brace shown without a cover and straps;

FIG. 4 is a rear perspective of a central panel of a mount of the cervical brace;

FIG. 5 is a rear perspective of a fastening system of the mount;

FIG. 6 is a rear perspective of the cervical brace in use; and

FIG. 7 is a front perspective of the cervical brace in use.

Corresponding reference characters indicate corresponding parts throughout the several views of the drawings.

### DESCRIPTION OF THE PREFERRED EMBODIMENT

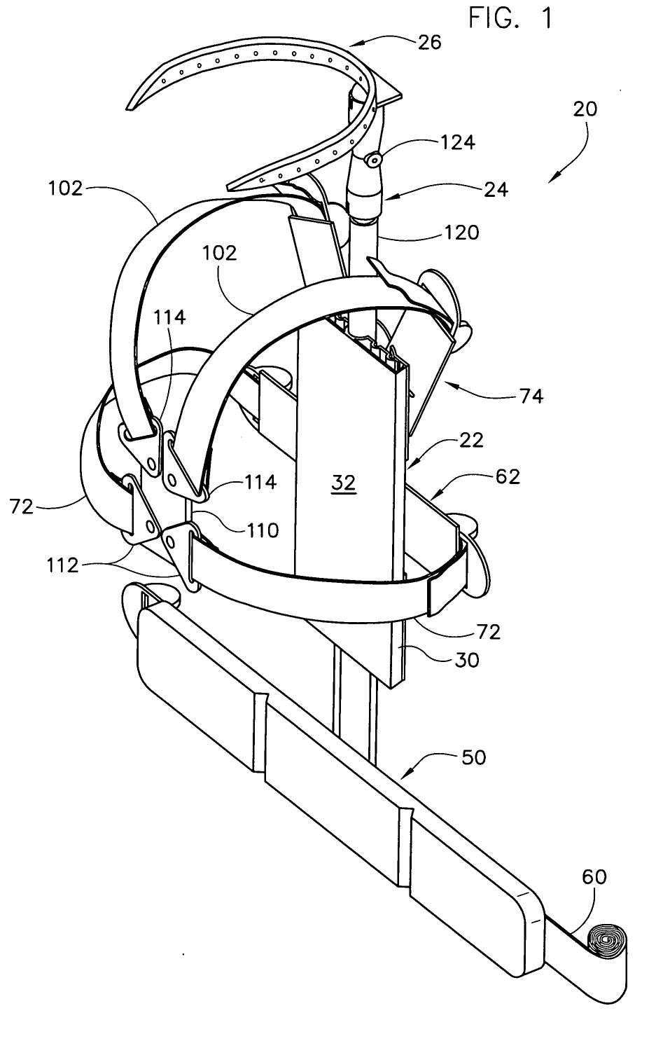

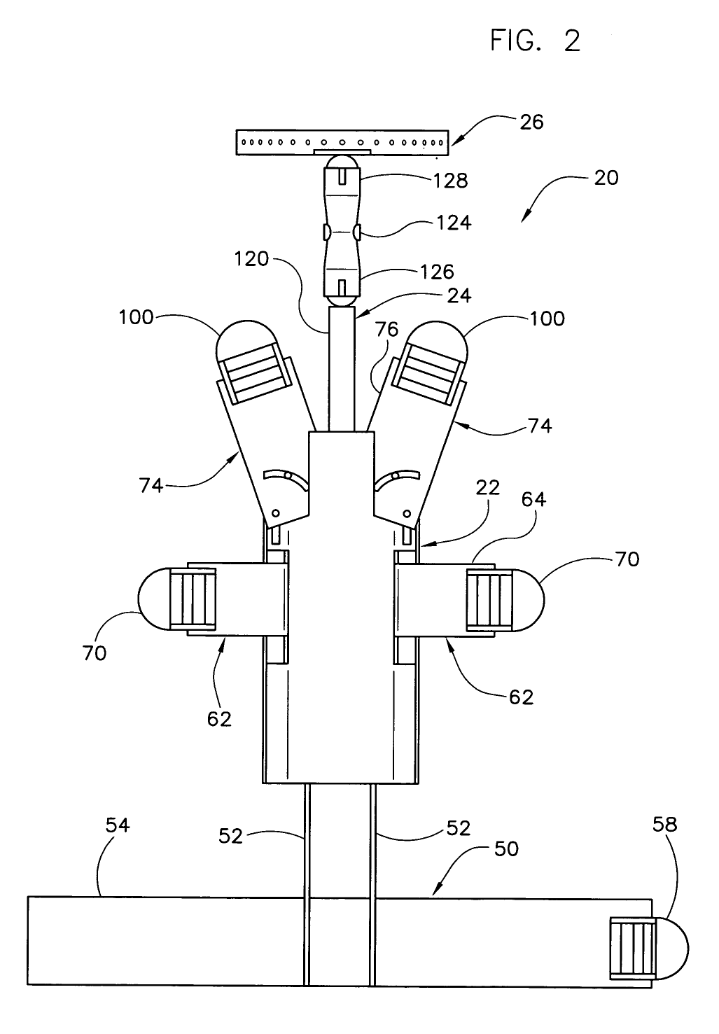

Referring now to the drawings and in particular to FIGS. 1 and 2, one embodiment of a cervical brace of the present invention is designated in its entirety by the reference numeral 20. The brace 20 generally comprises a mount (generally designated by 22) for attaching the brace to the chest and the abdomen of the patient, a support (generally designated by 24) extending upward from the mount to a position in use adjacent a rearward surface of the head of the patient, and a loop (generally designated by 26) sized and shaped for receiving the head of the patient. As illustrated in FIG. 1, the loop 26 is cantilevered from the support 24.

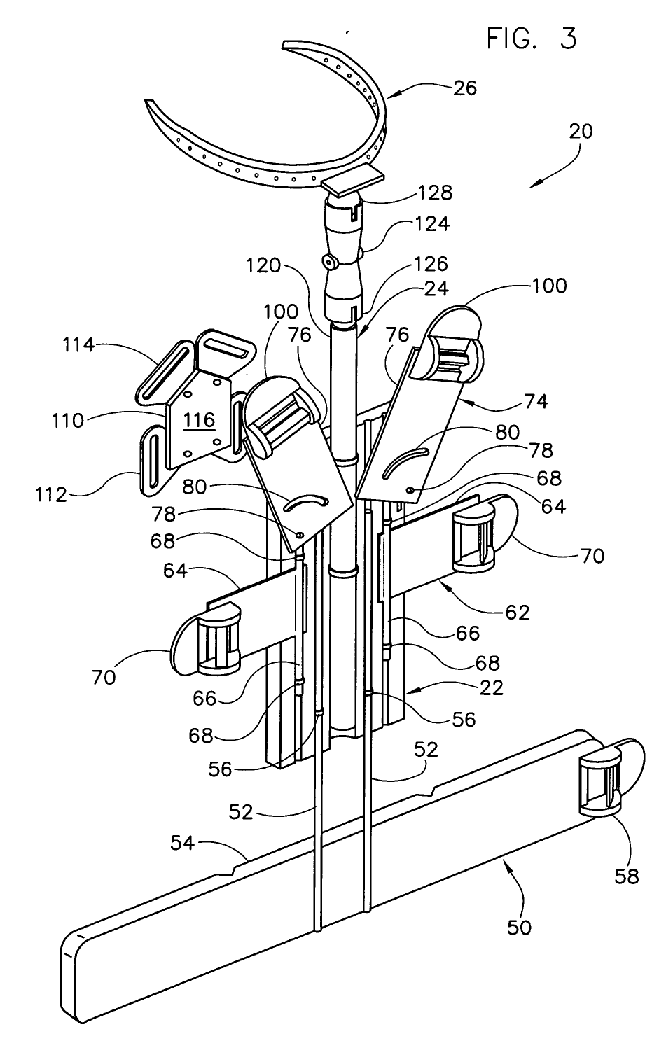

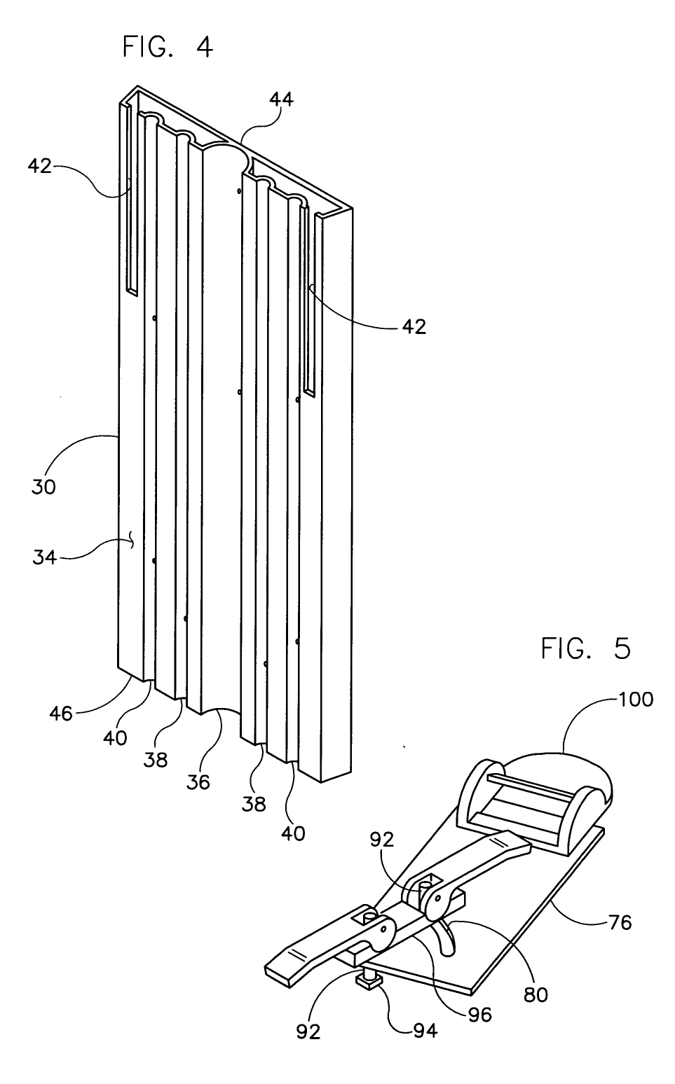

As illustrated in FIG. 3, the mount 22 includes a central panel 30 having a generally flat forward face 32 (FIG. 1) and a rearward face 34 opposite the forward face. As shown in FIG. 4, the rearward face 34 has a large central groove 36 extending vertically. Although the groove 36 may have other shapes without departing from the scope of the present invention, in one embodiment the groove 36 has a semi-elliptical cross section. A first pair of grooves 38 extend vertically along the rearward face 34 of the panel 30 so one groove is positioned on each side of the central groove 36. A second pair of grooves 40 extend vertically along the rearward face 34 of the panel 30 so one groove is positioned outside the first pair of grooves 38. Although the grooves 38, 40 may have other shapes without departing from the scope of the present invention, in one embodiment they have semi-circular cross sections. Two slots 42 are formed in the rearward face 34 of the central panel 30 outboard from the second pair of grooves 40 so they extend vertically adjacent an upper end 44 of the panel. The panel 30 has a lower end 46 opposite the upper end 44.

As shown in FIG. 3, the mount 22 also includes a waist prop (generally designated by 50) comprising a pair of rods 52 corresponding to the first pair of grooves 38 in the panel 30 and a flexible pad 54 mounted across lower ends of the rods. Although the rods 52 may be held in position in the corresponding grooves 38 by other means without departing from the scope of the present invention, in one embodiment the rods are held in position by strap clamps 56 having fasteners (e.g., cam-type fasteners or screw fasteners, not shown) so the pad 54 is positioned below the lower end 46 of the panel 30. A conventional webbing clasp 58 is provided on one end of the pad 54 for receiving webbing 60 (FIG. 1) to fasten the brace 20 to the patient as will be explained below. The mount 30 also includes a chest cinch (generally designated by 62) comprising flexible wings 64. A rod 66 is mounted on an inboard end of each wing 64. Each rod 66 is received in the corresponding groove 40 in the panel 30. Strap clamps 68 mounted adjacent the grooves 40 hold the rods 66 in place on the panel 30. A clasp 70 is positioned at an outboard end of each wing 64 for receiving for receiving webbing 72 (FIG. 1) to fasten the brace 20 to the patient as will be explained below. In addition, the mount 30 includes a shoulder cinch (generally designated by 74) comprising flexible wings 76. Openings 78, 80 are formed near the inboard end of each wing 76. As shown in FIG. 5, the openings 78, 80 receive a fastening system, generally designated by 90, mounted in the slots 42 in the panel 30 to hole the wings 76 in position.

The fastening system 90 includes a pair of studs 92 having heads 94 on one end. The heads 94 of the studs 92 are received in the slots 42 in the panel 30. A spacer 96 having holes (not shown) therein is assembled over the wings 76 so the studs 92 extend through the holes. A levered cam 98 is pivotally mounted to each stud 92 on an end opposite the head 94. When the cam 98 is in an unlocked position (not shown), the stud 92 is extending into the slot 42 of the panel 30 so the wing 76 is free to be positioned anywhere along the slot. When the cam 98 is in a locked position as shown, the stud 92 is retracted so the head 94 engages the slot 42 to hold the wing in position. Because one opening 80 in each wing is arcuate, an angle at which the shoulder cinch 74 extends from the panel 30 may be adjusted while the cam 98 is in the unlocked position.

As shown in FIG. 3, a clasp 100 is positioned at an outboard end of each wing 76 for receiving for receiving webbing 102 (FIG. 1) to fasten the brace 20 to the patient as will be explained below. Moreover, the mount 22 includes a breast plate 110 having rings 112, 114 for receiving the webbing 72, 102. As will be appreciated by those skilled in the art, the forward face 32 of the panel 30 and a rearward face 116 of the breast plate 90 may be padded (e.g., with lambs wool) without departing from the scope of the present invention.

As further shown in FIG. 3, the support 24 includes a vertical tube 120 that is received by the central groove 36 in the central panel 30 of the mount 22. Although the tube 120 may be held in position in the groove 36 by other means without departing from the scope of the present invention, in one embodiment the tube is held in position along the groove by strap clamps 122 having fasteners (e.g., cam-type fasteners or screw fasteners, not shown). Further, although the tube 120 may have other cross-sectional shapes without departing from the scope of the present invention, in one embodiment the tube has an oblong, oval or elliptical cross section so the tube resists turning in the groove 36 of the mount 22. An extender 124 having a ball joint 126, 128 at each end is mounted to an upper end of the tube 120. The loop 26 is mounted on the upper ball joint 128 of the extender 124 so the position and orientation of the loop can be adjusted relative to the support 24.

The loop 26 is a typical halo having an open side. In many conventional applications of this type of halo, the open side faces rearward. However, the loop 26 of the preferred embodiment is oriented so the open side faces forward to reduce obstruction of vision and interference with of lifestyle caused by the loop. Although other loops may be used without departing from the scope of the present invention, in one embodiment the loop 26 is a conventional loop available from PMT Corporation of Chanhassen, Minn. The loop 26 includes threaded holes (not shown) positioned for receiving threaded studs (not shown) for piercing the patient's skin and engaging the patients skull to rigidly hold the skull.

The entirety of the cervical brace 20 may be constructed of any combination of sufficiently stiff and strong materials. Preferably, the materials do not impede magnetic resonance imaging. Examples of such materials include fiberglass, nylon, and lambskin for the mount 22, woven carbon fiber/epoxy composite material, plastics, titanium, and elastomers for the support 24, and carbon fiber/epoxy composite material for the loop 26. Titanium is an example of a material that could be used in the various fasteners of the brace 20. Those skilled in the art will appreciate how the various components may be made using conventional materials.

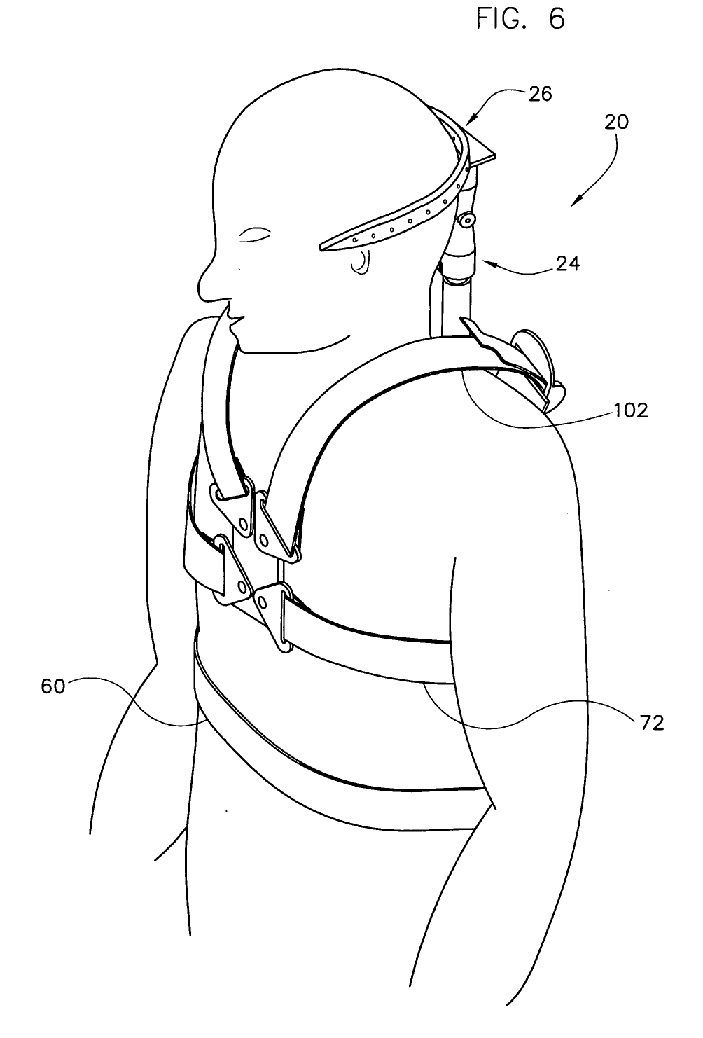

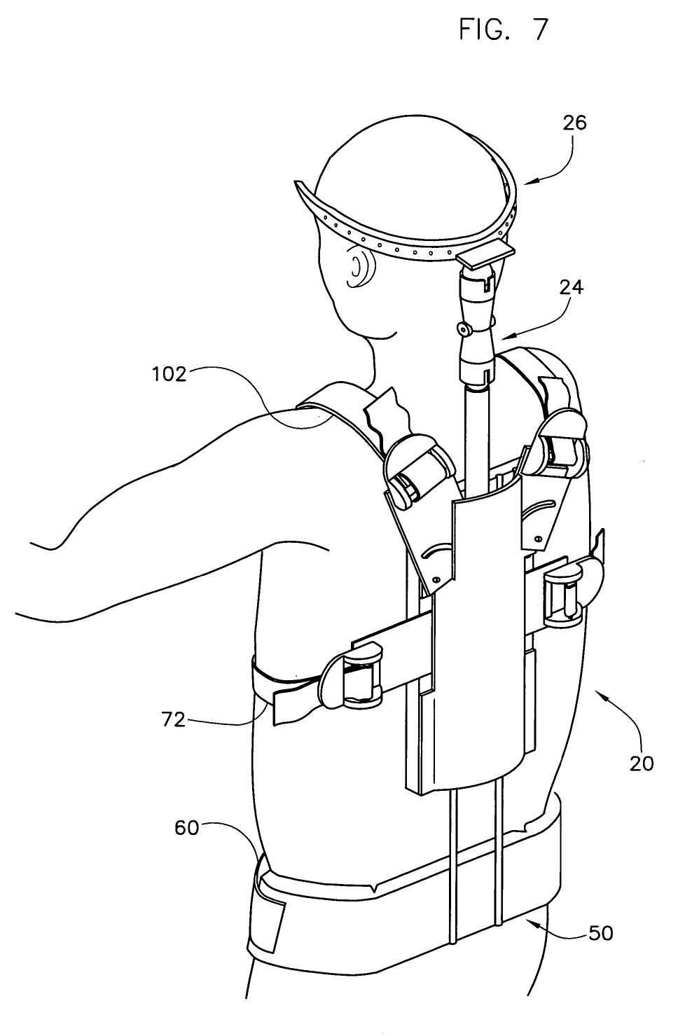

To use the brace 20, the positions of the waist prop 50, chest the cinches 62 and the shoulder cinches 74 are adjusted by loosening the corresponding strap clamps 56, 68 and cams 98 and moving the components to the desired locations relative to the central panel 30 so the mount 22 fits the patient. Once the prop 50 and cinches 62, 74 are in the desired locations, the clamps 56, 68 and cams 98 are tightened to hold the components in position. The breast plate 110 is positioned over the breast of the patient as shown in FIG. 6 and the webbing 72, 102 is threaded through the corresponding clasps, 70, 100. The webbing 72, 102 is tightened to hold the mount 22 in position on the patient. Because the breast plate 110 does not interfere with movement of the patient's diaphragm, the brace 20 does not move significantly as the patient breaths.

The height of the support 24 is adjusted by loosening the strap clamps 122 and moving the tube 120 into position. Once properly positioned, the strap clamps 122 are tightened. A cover 130 may be positioned over the strap clamps 56, 68, 122 to prevent them from being inadvertently loosened and adjusted. Once the tube 120 is in position, the position of the loop 26 may be adjusted by loosening the ball joints 126, 128 on the extender 124, moving the loop into position and tightening the joints. The threaded studs (not shown) may be screwed into the loop 26 and the patient's head in a conventional manner to hold the patient's head in position relative to the mount 22 and the patient's spine. In this way, the patient's spine is substantially immobilized.

The cervical brace 20 of the embodiment described above has several advantages over prior designs while providing portable stability and allowing free jaw movement. The brace 20 is held firmly in place and restrained from substantial movement relative to the patient's chest, abdomen and spine. Thus, the brace 20 of the present invention effectively restrains movement of the patient's head relative to the chest and/or abdomen. Moreover, the brace 20 does not substantially obstruct the patient's view during use or protrude in such a way as to unnecessarily inhibit an ordinary lifestyle. As a result, patients can wear most off-the-shelf shirts when wearing the cervical brace 20 of the present invention. Still further, the number of screws needed at each adjustment point have been reduced compared to conventional designs to reduce the time required to install the brace 20 and the difficulty encountered when installing the brace. In addition, the brace 20 of the embodiment described above can fit patients having uneven shoulders.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

As various changes could be made in the above constructions without departing from the scope of the invention, it is intended that all matter contained in the above description or shown in the accompanying drawings shall be interpreted as illustrative and not in a limiting sense.

1. A cervical brace for restraining movement of a head and neck vertebra of a patient relative to at least one of a chest and an abdomen of the patient, said brace comprising:
a mount sized and shaped for attachment to at least one of the chest and the abdomen of the patient;
a support extending upward from the mount to a position in use adjacent a rearward surface of the head of the patient; and
a loop sized and shaped for receiving the head of the patient cantilevered from the support,
wherein the support includes at least one joint for adjusting at least one of a pitch, a yaw and a roll of the loop relative to the mount.

2. A cervical brace as set forth in claim 1 wherein the joint comprises a ball and socket joint for adjusting the pitch, the yaw and the roll of the loop relative to the mount.

3. A cervical brace as set forth in claim 1 further comprising a plurality of straps extending from the mount for attaching the mount to at least one of the chest and the abdomen of the patient.

4. A cervical brace as set forth in claim 3 wherein the plurality of straps include a belt for at least partially circumscribing a waist of the patient.

5. A cervical brace as set forth in claim 3 wherein the plurality of straps include a first pair of straps positioned for extending over each shoulder of the patient, and a second pair of straps positioned for extending beneath each arm of the patient, said first and second pairs of straps being joined adjacent a breast of the patient.

6. A cervical brace as set forth in claim 1 wherein the loop is open at the front of the head of the patient.

7. A cervical brace for restraining movement of a head and neck vertebra of a patient relative to at least one of a chest and an abdomen of the patient, said brace comprising:
a mount sized and shaped for attachment to at least one of the chest and the abdomen of the patient;
a strap extending from the mount for attaching the mount to at least one of the chest and the abdomen of the patient
a support extending from the mount to a position in use adjacent a rearward surface of the head of the patient; and
a loop sized and shaped for receiving the head of the patient cantilevered from the support.

8. A cervical brace as set forth in claim 7 wherein the support includes at least one joint for adjusting at least one of a pitch, a yaw and a roll of the loop relative to the mount.

9. A cervical brace as set forth in claim 8 wherein the joint comprises a ball and socket joint for adjusting the pitch, the yaw and the roll of the loop relative to the mount.

10. A cervical brace as set forth in claim 7 wherein the strap includes a belt for at least partially circumscribing a waist of the patient.

11. A cervical brace as set forth in claim 7 wherein said strap is a first strap of a plurality of straps including a first pair of straps positioned for extending over each shoulder of the patient, and a second pair of straps positioned for extending beneath each arm of the patient, said first and second pairs of straps being joined adjacent a breast of the patient.

12. A cervical brace as set forth in claim 7 wherein the loop is open at the front of the head of the patient.

13. A cervical brace for restraining movement of a head and neck vertebra of a patient relative to at least one of a chest and an abdomen of the patient, said brace comprising:
a mount sized and shaped for attachment to at least one of the chest and the abdomen of the patient;
a support extending from the mount to a position in use adjacent a rearward surface of the head of the patient; and
a U-shaped loop sized and shaped for receiving the head of the patient cantilevered from the support.

14. A cervical braced as set forth in claim 13 wherein the loop is open at the front of the head of the patient in use.

15. A cervical brace as set forth in claim 13 wherein the support includes at least one joint for adjusting at least one of a pitch, a yaw and a roll of the loop relative to the mount.

16. A cervical brace as set forth in claim 15 wherein the joint comprises a ball and socket joint for adjusting the pitch, the yaw and the roll of the loop relative to the mount.

17. A cervical brace as set forth in claim 13 further comprising a plurality of straps extending from the mount for attaching the mount to at least one of the chest and the abdomen of the patient.

18. A cervical brace as set forth in claim 17 wherein the plurality of straps include a belt for at least partially circumscribing a waist of the patient.

19. A cervical brace as set forth in claim 17 wherein the plurality of straps include a first pair of straps positioned for extending over each shoulder of the patient, and a second pair of straps positioned for extending beneath each arm of the patient, said first and second pairs of straps being joined adjacent a breast of the patient.
